(19) United States
(12) Patent Application Publication
(10) Pub. No.: US 2015/0190194 A1
(43) Pub. Date: Jul. 9, 2015

(54) SYMPATHETIC NERVE ABLATION DEVICES WITH AN EXPANSION-LIMITING MEMBER

(71) Applicant: BOSTON SCIENTIFIC SCIMED, INC., MAPLE GROVE, MN (US)

(72) Inventors: JAN WEBER, MAASTRICHT (NL); MARTIN R. WILLARD, BURNSVILLE, MN (US); JEFFREY S. LINDQUIST, MAPLE GROVE, MN (US)

(73) Assignee: BOSTON SCIENTIFIC SCIMED, INC., MAPLE GROVE, MN (US)

(21) Appl. No.: 14/589,516

(22) Filed: Jan. 5, 2015

Related U.S. Application Data
(60) Provisional application No. 61/924,123, filed on Jan. 6, 2014.

(51) Int. Cl. A61B 18/14

(52) U.S. Cl. CPC A61B 18/1492; A61B 2018/00434

(57) ABSTRACT

Medical devices for sympathetic nerve modulation are disclosed. An example medical device for sympathetic nerve modulation may include a catheter shaft having a distal region. A compliant balloon may be coupled to the distal region. A flexible circuit assembly may be coupled to the compliant balloon. The flexible circuit assembly may include one or more electrodes. An expansion-limiting member may be coupled to the compliant balloon.

### CROSS-REFERENCE TO RELATED APPLICATIONS

This application claims priority under 35 U.S.C. §119 to U.S. Provisional Application Ser. No. 61/924,123, filed Jan. 6, 2014, the entirety of which is incorporated herein by reference.

### TECHNICAL FIELD

The present disclosure pertains to medical devices, and methods for using and manufacturing medical devices. More particularly, the present disclosure pertains to medical devices and methods that relate to sympathetic nerve modulation.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device for sympathetic nerve modulation may include a catheter shaft having a distal region. A compliant balloon may be coupled to the distal region. A flexible circuit assembly may be coupled to the compliant balloon. The flexible circuit assembly may include one or more electrodes. An expansion-limiting member may be coupled to the compliant balloon.

Another example medical device for sympathetic nerve modulation may include a catheter shaft having a distal region. A compliant balloon may be coupled to the distal region. A plurality of expansion-limiting fibers may be coupled to the compliant balloon. The expansion-limiting fibers may be capable of limiting the radial expansion of the compliant balloon to a pre-determined maximum diameter. The expansion-limiting fibers may be designed to shift between a first wavy configuration and a second substantially linear configuration. One or more pairs of bipolar electrodes may be coupled to the compliant balloon.

An example method for modulating sympathetic nerves may include providing a renal nerve ablation device. The renal nerve ablation device may include a catheter shaft having a distal region, a compliant balloon coupled to the distal region, and a plurality of expansion-limiting fibers coupled to the compliant balloon. The expansion-limiting fibers may be capable of limiting the radial expansion of the compliant balloon to a pre-determined maximum diameter. The expansion-limiting fibers may be designed to shift between a first wavy configuration and a second substantially linear configuration. The renal nerve ablation device may also include one or more pairs of bipolar electrodes coupled to the compliant balloon. The method may also include advancing the renal nerve ablation device through a blood vessel to a position within a renal artery, expanding the compliant balloon, and activating at least one of the one or more pairs of bipolar electrodes.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an example sympathetic nerve modulation system;

FIG. 2 is a side view of an example medical device disposed in a body lumen;

FIG. 3 illustrates an example flexible circuit assembly;

FIG. 4 is a side view of an example medical device including the flexible circuit assembly shown in FIG. 3;

FIG. 5 is a side of the example medical device shown in FIG. 4 in an expanded configuration;

FIG. 6 is a schematic view of a portion of an example medical device in a first configuration;

FIG. 7 is a schematic view of a portion of an example medical device in a second configuration;

FIG. 8 is a schematic view of a portion of an example medical device in a first configuration;

FIG. 9 is a schematic view of a portion of an example medical device in a second configuration;

FIG. 10 is a schematic view of a portion of an example medical device in a third configuration;

FIG. 11 is a schematic view of a portion of an example medical device in a fourth configuration;

FIGS. 12-13 illustrate an example method and/or components for manufacturing a substrate for use in an electrode assembly;

FIG. 14 is a schematic view of a portion of an example medical device in a first configuration;

FIG. 15 is a schematic view of a portion of an example medical device in a second configuration;

FIG. 16 is a schematic view of a portion of an example medical device in a first configuration; and

FIG. 17 is a schematic view of a portion of an example medical device in a second configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Certain treatments are aimed at the temporary or permanent interruption or modification of select nerve function. In some embodiments, the nerves may be sympathetic nerves. One example treatment is renal nerve ablation, which is sometimes used to treat conditions such as or related to hypertension, congestive heart failure, diabetes, or other conditions impacted by high blood pressure or salt retention. The kidneys produce a sympathetic response, which may increase the undesired retention of water and/or sodium. The result of the sympathetic response, for example, may be an increase in blood pressure. Ablating some of the nerves running to the kidneys (e.g., disposed adjacent to or otherwise along the renal arteries) may reduce or eliminate this sympathetic response, which may provide a corresponding reduction in the associated undesired symptoms (e.g., a reduction in blood pressure).

Some embodiments of the present disclosure relate to a power generating and control apparatus, often for the treatment of targeted tissue in order to achieve a therapeutic effect. In some embodiments, the target tissue is tissue containing or adjacent to nerves. In other embodiments, the target tissue is sympathetic nerves, including, for example, sympathetic nerves disposed adjacent to blood vessels. In still other embodiments the target tissue is luminal tissue, which may further comprise diseased tissue such as that found in arterial disease.

Many of the devices and methods described herein are discussed relative to renal nerve ablation and/or modulation. However, it is contemplated that the devices and methods may be used in other treatment locations and/or applications where sympathetic nerve modulation and/or other tissue modulation including heating, activation, blocking, disrupting, or ablation are desired, such as, but not limited to: blood vessels, urinary vessels, other body lumens or openings, or in other tissues via trocar and cannula access. For example, the devices and methods described herein can be applied to hyperplastic tissue ablation, cardiac ablation, pain management, pulmonary vein isolation, pulmonary vein ablation, tumor ablation, benign prostatic hyperplasia therapy, nerve excitation or blocking or ablation, modulation of muscle activity, hyperthermia or other warming of tissues, etc.

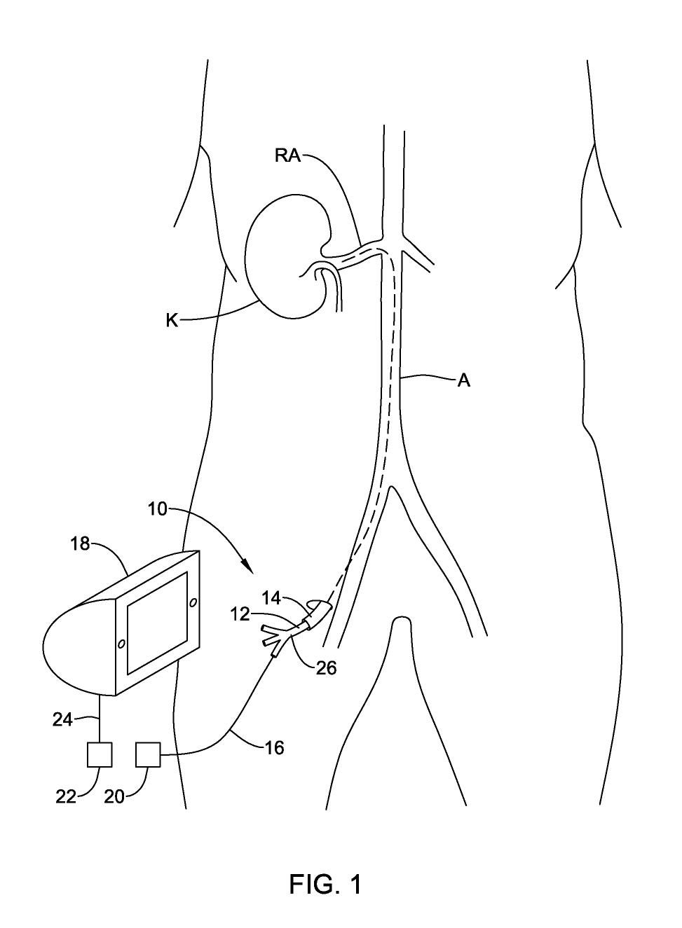

FIG. 1 is a schematic view of an example sympathetic nerve ablation system 10. System 10 may include a sympathetic nerve modulation and/or ablation device 12. Sympathetic nerve ablation device 12 may be used to ablate nerves (e.g., renal nerves) disposed adjacent to the kidney K (e.g., renal nerves disposed about a renal artery RA). In use, sympathetic nerve ablation device 12 may be advanced through a blood vessel such as the aorta A to a position within the renal artery RA. This may include advancing sympathetic nerve ablation device 12 through a guide sheath or catheter 14. When positioned as desired, sympathetic nerve ablation device 12 may be activated to activate one or more electrodes (not shown). This may include operatively coupling sympathetic nerve ablation device 12 to a control unit 18, which may include an RF generator, so as to supply the desired activation energy to the electrodes. For example, sympathetic nerve ablation device 12 may include a catheter shaft 26 and a wire or conductive member 16 coupled to catheter shaft 26. Conductive member 16 may include a first connector 20 that can be connected to a second connector 22 on the control unit 18 and/or a wire 24 coupled to the control unit 18. In at least some embodiments, the control unit 18 may also be utilized to supply/receive the appropriate electrical energy and/or signal to activate one or more sensors disposed at or near a distal end of sympathetic nerve ablation device 12. When suitably activated, the one or more electrodes may be capable of ablating tissue (e.g., sympathetic nerves) as described below and the one or more sensors may be used to detect desired physical and/or biological parameters.

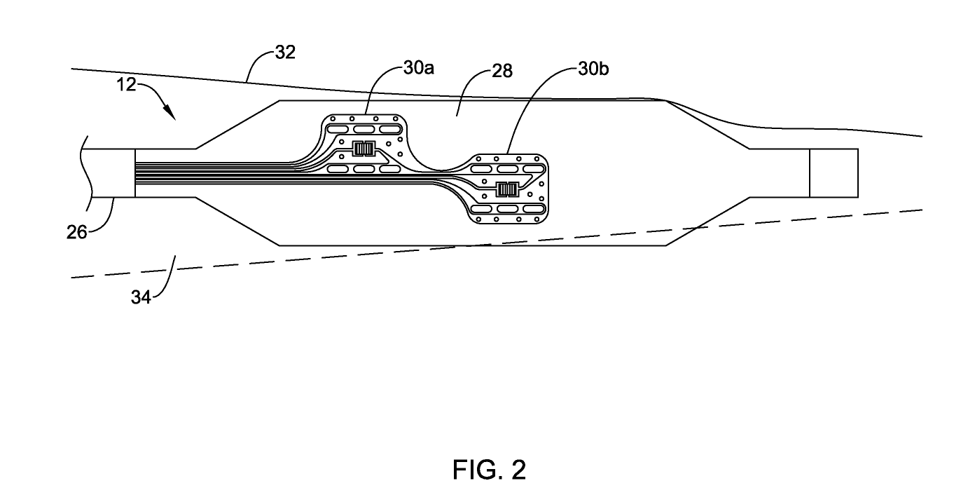

FIG. 2 illustrates sympathetic nerve ablation device 12 disposed within a lumen 34 of a vessel 32. In particular, device 12 includes catheter shaft 26 having an expandable member 28 attached thereto. In the illustrated embodiment, expandable member 28 may be a non-compliant balloon. Device 12 further includes two electrode assemblies 30a and 30b disposed on an external surface of non-compliant balloon 28.

Some blood vessels may have a generally tapered or narrowing shape. Because of this, during a medical procedure portions of non-compliant balloon 28 may contact the vessel wall, while other portions, particularly, proximal portions, may be spaced from the vessel wall, thereby leading to mal-apposition of non-compliant balloon 28 at the proximal portion. In the areas where the portions of non-compliant balloon 28 is in contact (e.g., narrowed areas), there may be an increased expansion force on the vessel, which could lead to stress induced trauma to the vessel 32.

The devices disclosed herein are designed to provide good apposition to the vessel wall while minimizing trauma to blood vessel. For example, the devices disclosed herein may utilize a compliant balloon. This may allow for increased contact between the electrodes on the balloon and the vessel wall. Furthermore, the devices disclosed herein may also include an expansion-limiting member. This may allow the devices to have a pre-determined maximum diameter and/or length. Accordingly, the devices disclosed herein may have some of the desirable characteristics of a compliant balloon (e.g., improved conformity with vessels of different sizes, reduced trauma, etc.), while also sharing some of the desirable characteristics of a non-compliant balloon (e.g., predictable maximum diameter and/or length, etc.).

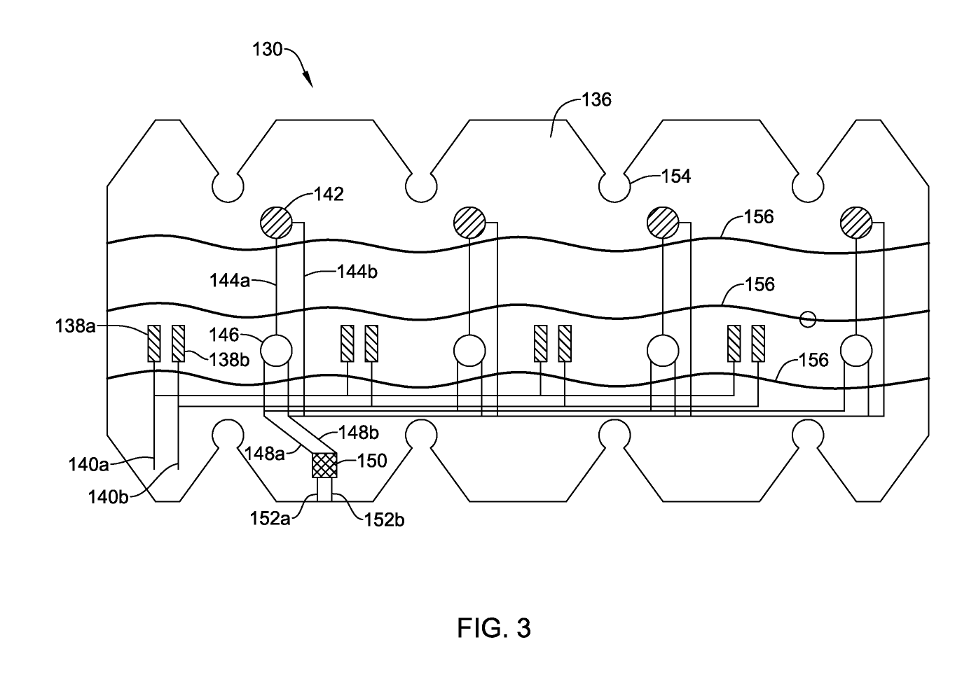

FIG. 3 illustrates an example flexible circuit assembly 130 that may be used with the sympathetic nerve ablation devices disclosed herein. Assembly 130 may include a substrate 136. A plurality of electrodes such as electrodes 138a/138b may be coupled to substrate 136. One or more leads such as leads 140a/140b may be coupled to electrodes 138a/138b. Electrodes 138a/138b may vary in form. In at least some embodiments, electrodes 138a/138b may be RF electrodes. Other types of electrodes are contemplated. For example, electrodes 138a/138b could be optical and/or laser electrodes, acoustic and/or ultrasound electrodes, resistive heating electrodes, and the like, or any other suitable type of electrodes. Leads 140a/140b may also vary in form. For example, leads 140a/140b may be conductive traces or wires disposed along flexible circuit assembly 130. In at least some embodiments, leads 140a/140b (and/or other leads disclosed herein) may have a wavy or sinusoidal configuration that is designed to accommodate stretching and/or expansion. For example, the wavy configuration of leads 140a/140b may allow leads 140a/140b to stretch when flexible circuit assembly 130 is utilized with an expandable balloon such as those disclosed herein.

Substrate 136 may also include one or more sensors. For example, substrate 136 may include one or more pressure sensors such as pressure sensors 142. In at least some embodiments, pressure sensor(s) 142 may be distal pressure sensors that are designed to measure pressure (and/or resistance to further inflation) along the distal region of assembly 130. One or more leads 144a/144b may be coupled to pressure sensor(s) 142. Substrate 136 may include one or more additional pressure sensors such as pressure sensors 146. In at least some embodiments, pressure sensor(s) 146 may be proximal pressure sensors that are designed to measure pressure (and/or resistance to further inflation) along the proximal region of assembly 130. One or more leads 148a/148b may be coupled to pressure sensor(s) 146. In at least some embodiments, an input/output unit 150 may be utilized to coordinate the connection of the various leads along substrate 136. One or more leads 152a/152b may be coupled to unit 150 and extend proximally therefrom and, ultimately, may be coupled to control unit 18. Substrate 136 may also be formed with a number of cutouts 154, which may allow assembly to be compactly wrapped around a structure with generally conical ends such as a balloon.

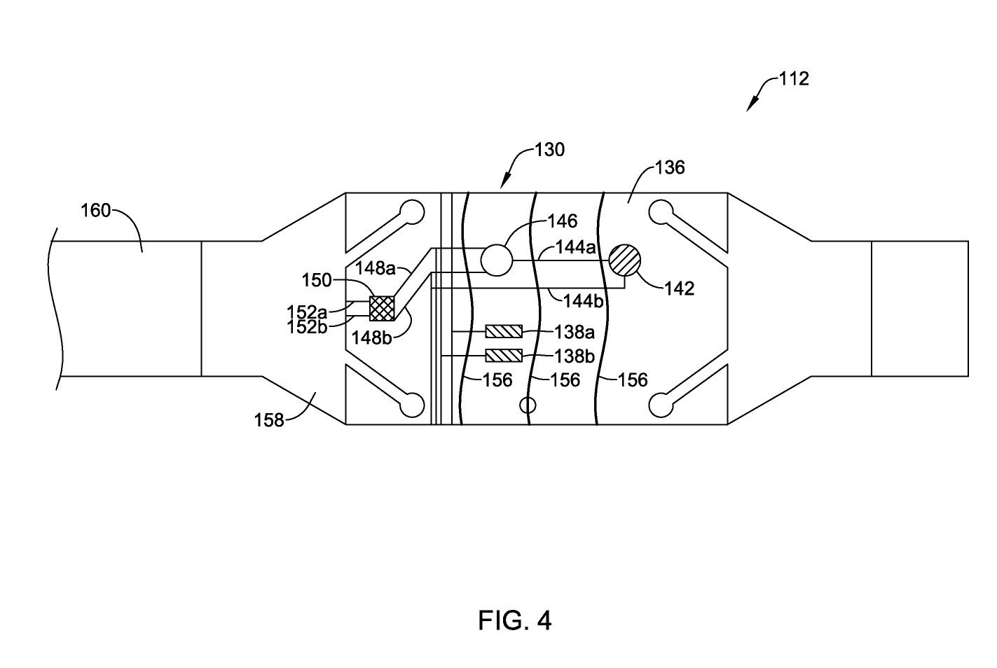

One or more expansion-limiting members 156 may be coupled to substrate 136. Expansion-limiting members 156, as the name suggests, may aid in limiting the amount of expansion that may occur when assembly 130 is coupled to an expandable member. For example, FIG. 4 illustrates an example medical device 112. Device 112 may include a catheter shaft 160 and an expandable member 158 coupled to catheter shaft 160. In at least some embodiments, expandable member 158 may be a balloon. In other embodiments, expandable member 158 may take the form of a basket or basket-like structure.

Expansion-limiting members 156 may be formed a number of different materials including those materials disclosed herein. For example, expansion-limiting members 156 may include an ultra-high molecular weight polyethylene (e.g., DYNEEMA), a polyimide (e.g., VESPEL, AURUM, P84, or the like), a polybenzimidazole (e.g., CELAZOLE), a polyamide-imide (e.g., TORLON), a polyetheretherketone (e.g., VICTREX, KADEL, or the like), a polytetrafluoroethylene (e.g., TEFLON, HOSTFLON, or the like), a polyphenylene sulfide (e.g., RYTON, FORTRON, THERMOCOMP, SUPEC, or the like), a polyetherimide (e.g., ULTEM), a polypthalamide (e.g., AMODEL, BGU, or the like), an aromatic polyamide (e.g., RENY, ZYTEL HTN, STANYL, or the like), a liquid crystal polymer (e.g., XYDAR, VECTRA, ZENITE, or the like), a nylon, fiberglass, a polyacetal, a polycarbonate, a polypropylene, a, acrylonitrile butadiene styrene, a polybutylene terephthalate, a polyurethane, a polyethylene terephthalate, a polyetheretherketone, or the like. These are just examples. Other materials are contemplated.

The number, arrangement, and distribution of expansion-limiting members 156 may vary. For example, assembly 130 may include one, two, three, four, five, six, seven, eight, or more expansion-limiting members 156. Expansion-limiting members 156 may be disposed “regularly” or otherwise evenly along substrate 133. Alternatively, a non-uniform distribution may be utilized. In some embodiments, expansion-limiting members 156 may extend along a full width or length of substrate 133. Alternatively, one or more expansion-limiting members 156 may span only a portion of the length or width of substrate 133. In some embodiments, expansion-limiting members 156 may be disposed or attached to an outer surface of substrate 133. Alternatively, expansion-limiting members 156 may be embedded within substrate 133. In still other embodiments, expansion-limiting members 156 may be defined by stamping a pattern into the material of substrate 133 (and/or the balloon material). In still other embodiments, expansion-limiting members 156 may be coupled or attached to an outer surface of a balloon. In still other embodiments, expansion-limiting members 156 may be embedded between two layers of a balloon.

Expansion-limiting members 156 may generally take the form of fibers that are distributed in a meandering, “zig-zag”, curved, wavy, sinusoidal, “s-shaped”, or other configuration. In general, the configuration of expansion-limiting members 156 is designed so that expansion-limiting members 156 can shift between an initial “wavy” configuration and a second substantially linear configuration. It can be appreciated that when expansion-limiting members 156 reaches a substantially linear configuration, further expansion of assembly 130 (and any underlying structures such as balloons) can be limited. When assembly 130 is used with a balloon, expansion-limiting members 156 may shift between the “wavy” configuration (which allows for balloon expansion in the radial direction) to a “linear” configuration (which substantially prevents further balloon expansion in the radial direction). Because expansion-limiting members 156 may define a pre-determined limit to balloon expansion, when expansion-limiting members 156 are used with a compliant balloon (e.g., which may have desirable wall apposition and/or conformability characteristics, lower inflation pressure needs, reduced trauma including reduced baro-trauma) they may allow the compliant balloon to also gain some of the desirable features of a non-compliant balloon (e.g., pre-determined limits on expansion).

As indicated above, a sinusoidal or sine-like pattern may be used for expansion-limiting members 156. In some embodiments, variations to the sinusoidal pattern may vary. For example, the shape of expansion-limiting members 156 may be altered so that the “amplitude” and/or “frequency” of the waves formed in expansion-limiting members 156 may vary. It can be appreciated that increasing the frequency while reducing the amplitude to the same relative amount results in waves with the same arc length (where the arc length may be mathematically defined). Because the arrangement of the waves may impact both radial expansion (due to a lengthening of expansion-limiting members 156) and also impact elongating in the axial direction, manipulation of the shape of the “waves” in expansion-limiting members 156 may allow for a desirable impact on both expansion and elongation. For example, increasing the frequency of the wave may not only desirably impact radial expansion, it may also help to limit axial elongation. While alteration to the “amplitude” and/or “frequency” are disclosed, other variations in the shape of expansion-limiting members 156 are contemplated.

Electrodes 138a/138b and/or the various other components of assembly 130 may be disposed along substrate 133 in a suitable member. For example, electrodes 138a/138b and/or the various lead disposed along substrate 133 may be electroplated, deposited, printed, or otherwise secured to substrate.

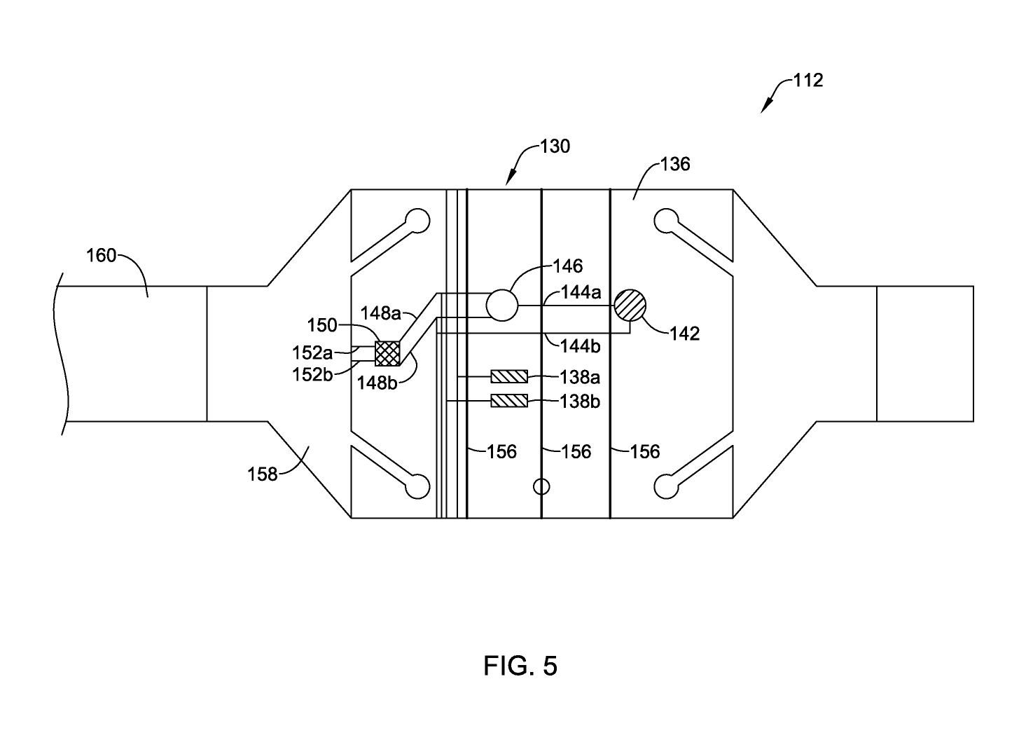

As suggested herein, assembly 130 may be disposed about balloon 158. When balloon 158 is in a non-inflated or partially inflated state (e.g., as shown in FIG. 4), expansion-limiting members 156 may have a “wavy” or non-linear configuration. In other words, the expansion-limiting members 156 may be in a configuration that allows for further expansion of balloon 158. Upon expansion of balloon 158 (e.g., as shown in FIG. 5), expansion-limiting members 156 may begin to straighten and, ultimately, may reach a point where expansion-limiting members 156 reach a substantially linear state and can no longer expand. At this point, expansion-limiting members 156 may pose a mechanical barrier to further expansion of balloon 158 and, thus, “limit” balloon expansion.

In at least some embodiments, balloon 158 may be a compliant balloon formed from a compliant polymer. Assembly 130 (e.g., substrate 133) may be formed from a compatible material. For example balloon 158 and substrate 133 may be formed from the same material. In some embodiments, substrate 133 may be formed from or otherwise include a foil or metallic material. The foil may serve as a substrate onto which a conductive trace or other electronic trace may be disposed. Other variations are contemplated including the use of any of the materials disclosed herein.

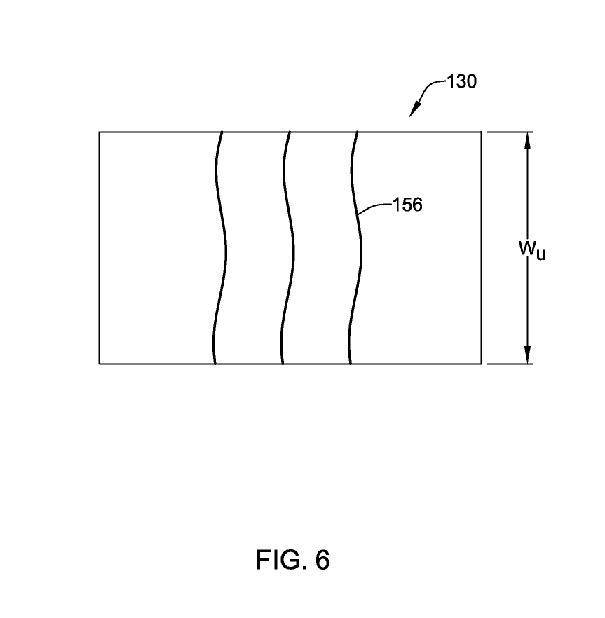

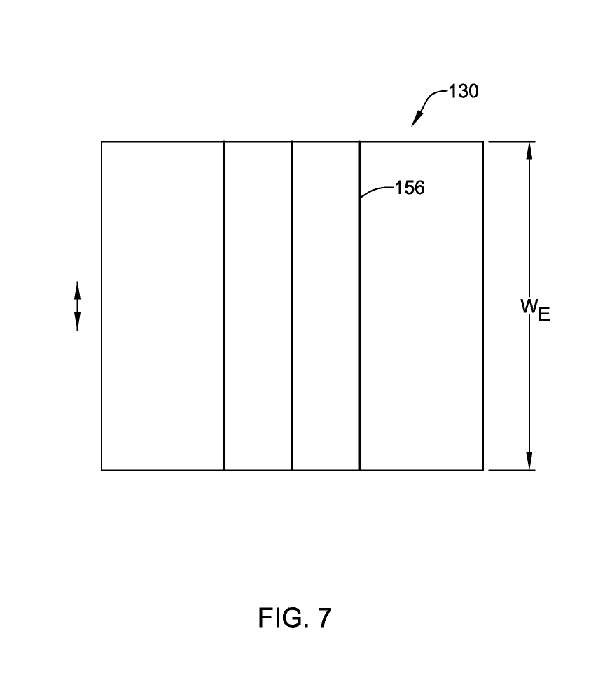

FIGS. 6-7 illustrate the use and/or function of expansion-limiting members 156. For example, FIG. 6 schematically shows assembly 130 with expansion-limiting members 156. In this example, assembly 130 is shown as having an unexpanded width Wᵤ. Expansion of assembly 130 (e.g., by expanding balloon 158), may cause assembly 130 to expand. When doing so, assembly 130 may have an expanded width Wₑ greater than the unexpanded width Wᵤ.

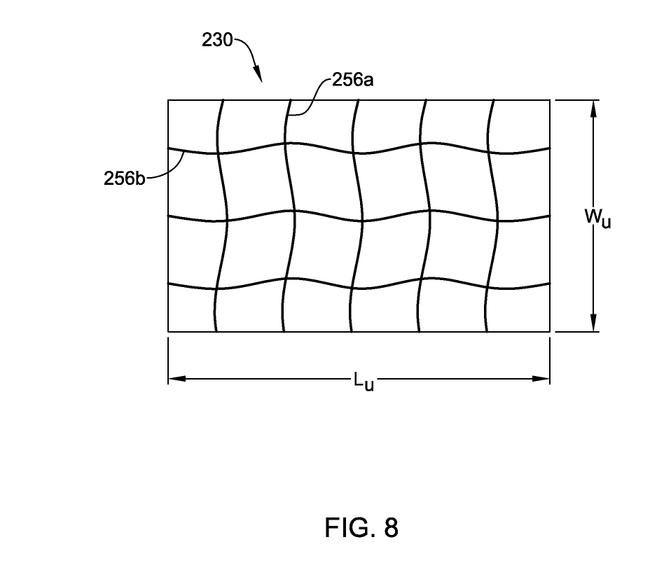

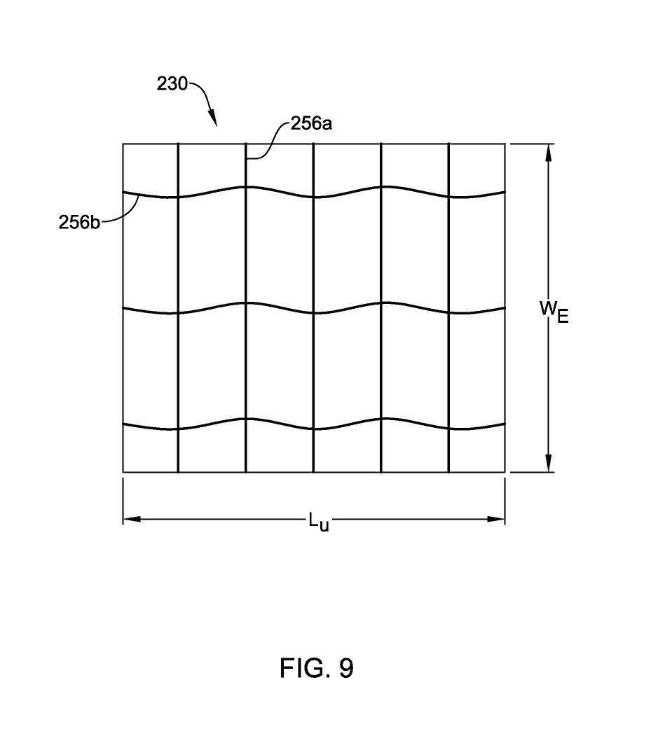

While the use of expansion-limiting members 156 may aid in limiting the radial expansion of balloon 158, it may also be desirable to limit the axial length of the electrode assembly. FIGS. 8-11 illustrate the use and/or function of expansion of another example electrode assembly 230. Assembly 230 may be similar in form and function to other assemblies disclosed herein. Assembly 230 may include a first set of expansion-limiting member 256a and a second set of expansion-limiting members 256b. According to this embodiment, expansion limiting members 256a may be designed to limit radial expansion. For example, when assembly 230 is radially expanded from an unexpanded width Wᵤ (e.g., as shown in FIG. 8) to an expanded width Wₑ (e.g., as shown in FIG. 9), expansion-limiting members 256a may limit any further expansion. When assembly 230 is utilized with an expandable balloon, expansion-limiting members 256a may limit radial expansion of the balloon.

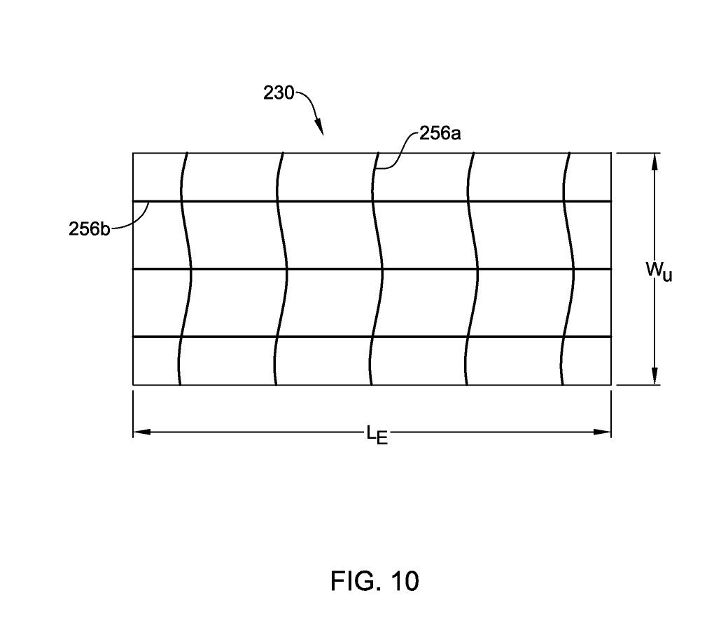

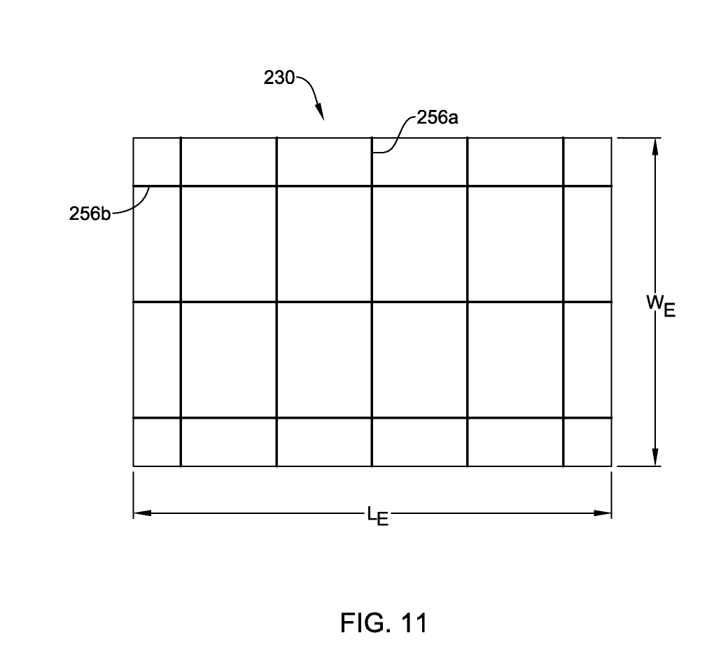

Expansion-limiting members 256b may be designed to limit axial elongation. For example, when assembly 230 is axially elongated from an unexpanded length Lᵤ (e.g., as shown in FIG. 8) to an expanded length Le (e.g., as shown in FIG. 10), expansion-limiting members 256b may limit any further elongation. When assembly 230 is utilized with an expandable balloon, expansion-limiting members 256b may limit axial elongation of the balloon. It can be appreciated that expansion-limiting members 256a/256b may be used together to simultaneously limit both radial expansion and axial elongation as shown in FIG. 11.

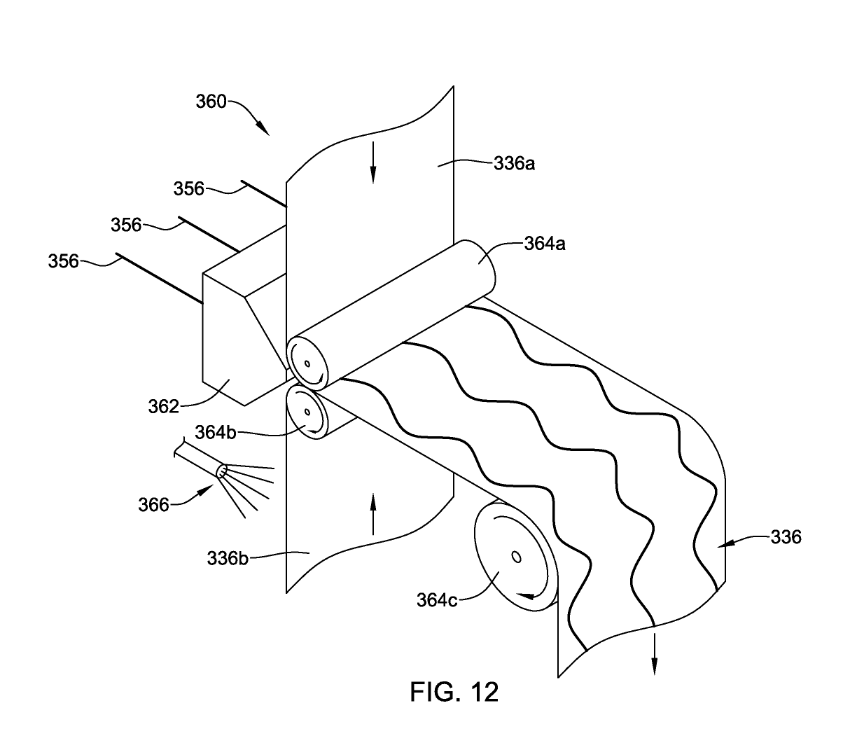

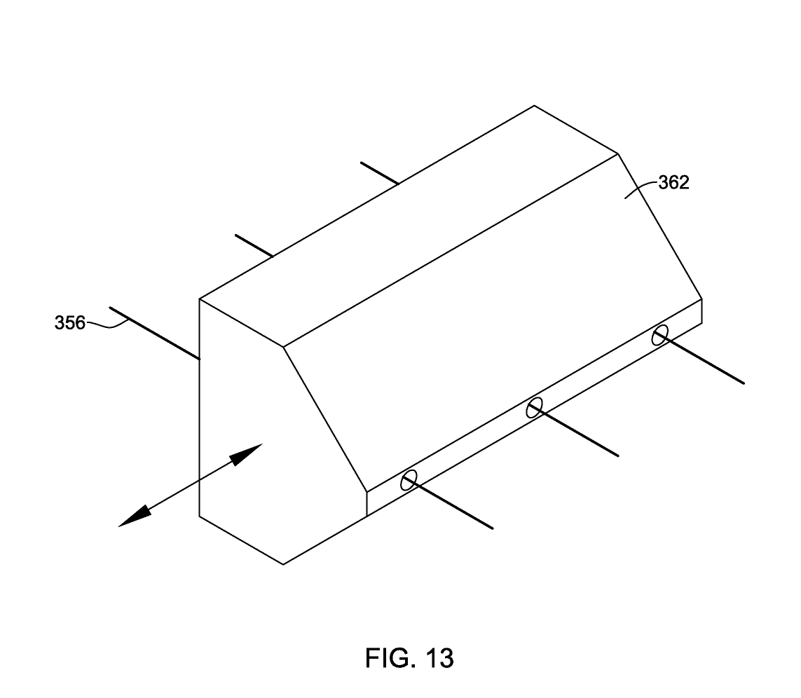

FIGS. 12-13 illustrate some of the methods and/or manufacturing components that may be utilized to form a medical device such as those devices disclosed herein. For example, FIG. 12 illustrates a manufacturing assembly 360 including a vibrating module 362. Module 362 may be used a guide for feeding expansion-limiting members 356 (e.g., the fibers used to make expansion-limiting members 356) into contact with substrate 336. For example, substrate 336 may be formed by combining two sleeves or portions 336a/336b. This may include applying an adhesive with a dispenser or sprayer 366 to one of the portions (e.g., portion 336b) and then feeding portions 336a/336b through a series of rollers 364a/364b/364c. While passing portions 336a/336b through rollers 364a/364b, vibrating module 362 may vibrated laterally (e.g., as depicted in FIG. 13) so that expansion-limiting members 356 move in a wave-like manner and are laminated between portions 336a/336b in a desired “wave-like” configuration.

Upon completion of the lamination process, substrate 336 may be cut to the desired dimensions and the remaining portions of the electrode assembly (electrodes, leads, sensors, etc.) may be coupled or otherwise attached thereto. When suitably configured, substrate 336 (e.g., which now may take the form of a fully-assembled electrode assembly) may be secured to, for example, a balloon.

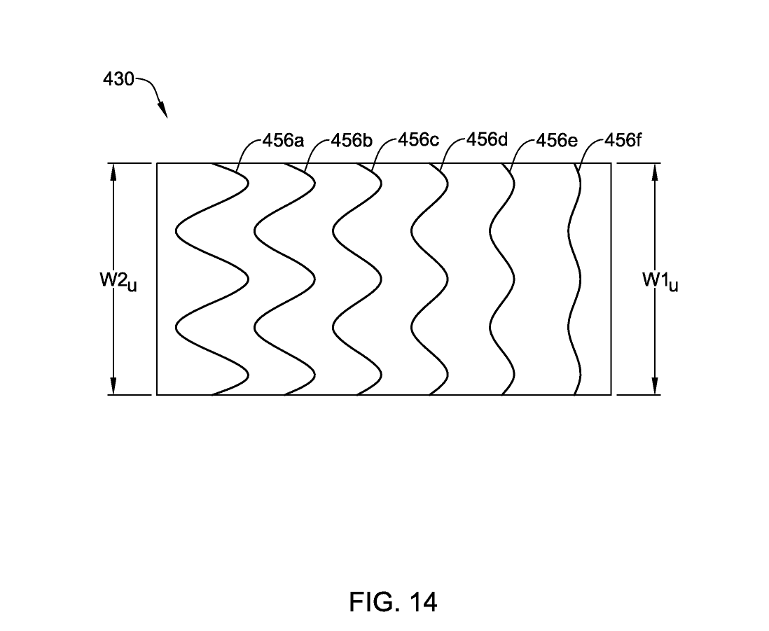

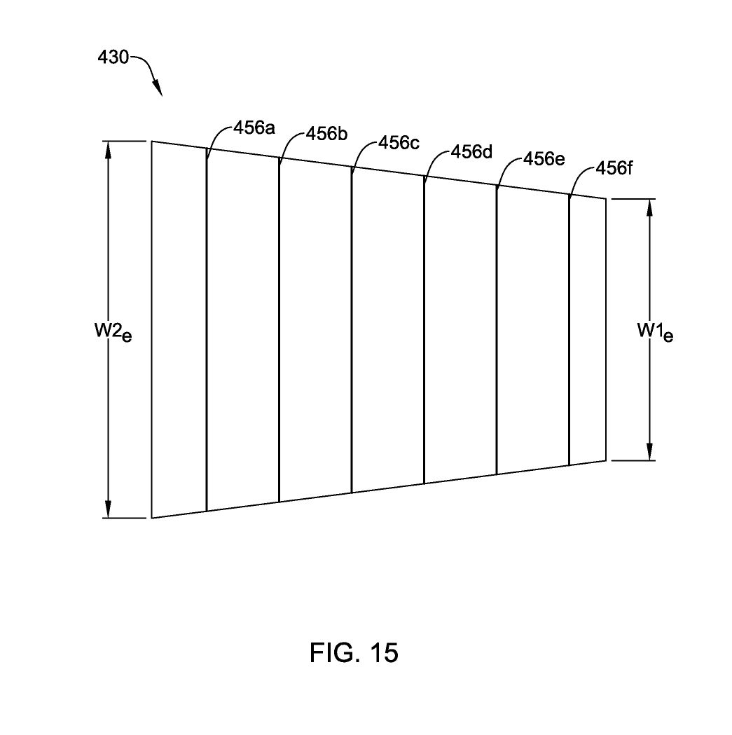

FIGS. 14-15 illustrate another example flexible circuit assembly 430 that may be used with the sympathetic nerve ablation devices disclosed herein. Assembly 430 may include a set of expansion-limiting members 456a/456b/456c/456d/456e/456f. Expansion-limiting members 456a/456b/456c/456d/456e/456f may have a wavy configuration where the amplitude of the “wave” varies. For example, expansion-limiting members 456a/456b/456c/456d/456e/456f may extend across the length of flexible circuit assembly 430 and longitudinally-adjacent expansion-limiting members 456a/456b/456c/456d/456e/456f may have differing amplitudes. Adjacent to expansion-limiting members with a “smaller amplitude” (e.g., expansion-limiting member 456f), the width of flexible circuit assembly 430 may expand less that at locations adjacent to expansion limiting members with a “larger amplitude” (e.g., expansion-limiting member 456a). For example, adjacent to expansion-limiting member 456f, flexible circuit assembly 430 may shift from an unexpanded width W1ᵤ as shown in FIG. 14 to an expanded width W1ₑ as shown in FIG. 15. Adjacent to expansion-limiting member 456a, flexible circuit assembly 430 may shift from an unexpanded width W2ᵤ as shown in FIG. 14 to an expanded width W2ₑ as shown in FIG. 15. W1ₑ may be smaller than W2ₑ, for example due to the decreasing amplitude and/or length of the expansion limiting members. This may result in a flexible circuit assembly 430 that, when used with a balloon, may constrain the balloon so that it expands to take a tapered shape.

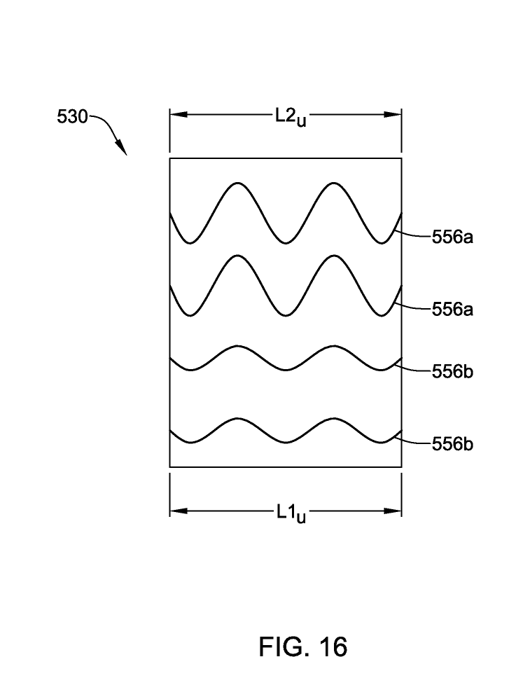

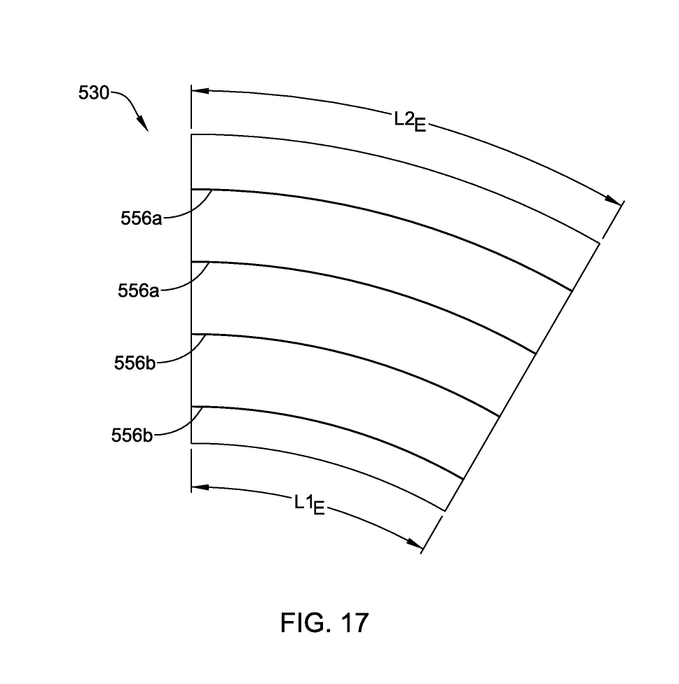

FIGS. 15-16 illustrate another example flexible circuit assembly 530 that may be used with the sympathetic nerve ablation devices disclosed herein. Assembly 530 may include a set of expansion-limiting members 556a/556b/556c/556d. Expansion-limiting members 556a/556b/556c/556d may have a wavy configuration where the amplitude of the “wave” varies across the width of flexible circuit assembly 530. For example, expansion-limiting members 556a/556b/556c/556d may extend across the width of flexible circuit assembly 530 and circumferentially-adjacent expansion-limiting members 556a/556b/556c/556d may have differing amplitudes. Adjacent to expansion-limiting members with a “smaller amplitude” (e.g., expansion-limiting member 556d), the length of flexible circuit assembly 530 may expand less that at locations adjacent to expansion limiting members with a “larger amplitude” (e.g., expansion-limiting member 556a). For example, adjacent to expansion-limiting member 556d, flexible circuit assembly 530 may shift from an unexpanded length L1ᵤ as shown in FIG. 16 to an expanded length L1ₑ as shown in FIG. 17. Adjacent to expansion-limiting member 556a, flexible circuit assembly 430 may shift from an unexpanded length L2ᵤ as shown in FIG. 16 to an expanded length L2ₑ as shown in FIG. 17. L1ₑ may be smaller than L2ₑ, for example due to the decreasing amplitude and/or length of the expansion limiting members. This may result in a flexible circuit assembly 530 that, when used with a balloon, may constrain the balloon so that it expands to take a curved or bending shape.

The materials that can be used for the various components of device 112 (and/or other devices disclosed herein) may include those commonly associated with medical devices. For simplicity purposes, the following discussion makes reference to device 112 and/or the components thereof. However, this is not intended to limit the devices and methods described herein, as the discussion may be applied to any of the other similar devices disclosed herein.

Device 112 and/or other components thereof may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, cellulose, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of device 112 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of device 112 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of device 112 to achieve the same result.

In some embodiments, a degree of Magnetic Resonance Imaging (MRI) compatibility is imparted into device. For example, device 112, or portions thereof, may be made of a material that does not substantially distort the image and create substantial artifacts (i.e., gaps in the image). Certain ferromagnetic materials, for example, may not be suitable because they may create artifacts in an MRI image. Device 112, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

U.S. patent application Ser. No. 13/750,879 filed on Jan. 25, 2013 is herein incorporated by reference.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device for sympathetic nerve modulation, comprising:
a catheter shaft having a distal region;
a compliant balloon coupled to the distal region;
a flexible circuit assembly coupled to the compliant balloon, the flexible circuit assembly including one or more electrodes; and
an expansion-limiting member coupled to the compliant balloon.

2. The medical device of claim 1, wherein the flexible circuit assembly includes one or more temperature sensors.

3. The medical device of claim 1, wherein the flexible circuit assembly includes one or more pressure sensors.

4. The medical device of claim 1, wherein the expansion-limiting member includes one or more fibers disposed about the compliant balloon that limit radial expansion of the compliant balloon.

5. The medical device of claim 4, wherein the one or more fibers are designed to shift from a first non-linear configuration to a second substantially linear configuration.

6. The medical device of claim 5, wherein the first configuration is a wavy configuration.

7. The medical device of claim 1, wherein the expansion-limiting member includes one or more fibers disposed about the compliant balloon that limit axial elongation of the compliant balloon.

8. The medical device of claim 1, wherein the expansion-limiting member includes a first set of fibers designed to limit radial expansion of the compliant balloon and a second set of fibers designed to limit axial elongation of the compliant balloon.

9. The medical device of claim 1, wherein the flexible circuit assembly includes a substrate attached to the compliant balloon, and wherein the compliant balloon and the substrate are formed from the same material.

10. The medical device of claim 1, wherein the flexible circuit assembly includes a foil that is wrapped about the compliant balloon.

11. The medical device of claim 1, wherein the expansion-limiting member is disposed along an outer surface of the compliant balloon.

12. The medical device of claim 1, wherein the expansion-limiting member is defined by stamping a pattern into a surface of the flexible circuit assembly.

13. The medical device of claim 1, wherein the expansion-limiting member embedded within the compliant balloon.

14. The medical device of claim 1, wherein a plurality of expansion-limiting members are coupled to the compliant balloon and wherein at least some of the plurality of expansion-limiting members have differing amplitudes.

15. A medical device for sympathetic nerve modulation, comprising:
a catheter shaft having a distal region;
a compliant balloon coupled to the distal region;
a plurality of expansion-limiting fibers coupled to the compliant balloon, the expansion-limiting fibers being capable of limiting the radial expansion of the compliant balloon to a pre-determined maximum diameter;
wherein the expansion-limiting fibers are designed to shift between a first wavy configuration and a second substantially linear configuration; and
one or more pairs of bipolar electrodes coupled to the compliant balloon.

16. The medical device of claim 15, further comprising a plurality of elongation-limiting fibers coupled to the compliant balloon, the elongation-limiting fibers being capable of limiting the axial elongation of the compliant balloon.

17. The medical device of claim 15, wherein the one or more pairs of bipolar electrodes are coupled to a flexible circuit assembly that is attached to the compliant balloon.

18. The medical device of claim 17, wherein the flexible circuit assembly includes a substrate attached to the compliant balloon, and wherein the compliant balloon and the substrate are formed from the same material.

19. The medical device of claim 17, wherein the flexible circuit assembly includes a foil that is wrapped about the compliant balloon.

20. A method for modulating sympathetic nerves, the method comprising:
providing a renal nerve ablation device, the renal nerve ablation device comprising:
a catheter shaft having a distal region,
a compliant balloon coupled to the distal region,
a plurality of expansion-limiting fibers coupled to the compliant balloon, the expansion-limiting fibers being capable of limiting the radial expansion of the compliant balloon to a pre-determined maximum diameter,
wherein the expansion-limiting fibers are designed to shift between a first wavy configuration and a second substantially linear configuration, and
one or more pairs of bipolar electrodes coupled to the compliant balloon;
advancing the renal nerve ablation device through a blood vessel to a position within a renal artery;
expanding the compliant balloon; and
activating at least one of the one or more pairs of bipolar electrodes.
